SEQUENCE LISTING (12 sequences)

SEQ ID NO: 1 - protein, 513 aa, synthetic construct
  REGION 1..513: note = Description of sequence: amino acid sequence of a truncated HBcAg protein 149
MDIDPYKEFGATVELLSFLPSDFFPSVRDLLDTASALYRDALESPEHASPHHTALRQAILCWGELMTLATWVGVNLEDGGGSDSATHIKFSKRDEDGRELAGATMELRDSSGKTISTWISDGHVKDFYLYPGKYTFVETAAPDGYEVATPIEFTVNEDGQVTVDGEATEGDAHTGGGSPASRDLVVSYVNTNMGLKFRQLLWFHISALTFGRETVIEYLVSFGVWIRTPPAYRPPNAPILSTLPETTVVGGSGGSGGSGGSGGSMDIDPYKEFGATVELLSFLPSDFFPSVRDLLDTASALYRDALESPEHASPHHTALRQAILCWGELMTLATWVGVNLEDGGGSDSATHIKFSKRDEDGRELAGATMELRDSSGKTISTWISDGHVKDFYLYPGKYTFVETAAPDGYEVATPIEFTVNEDGQVTVDGEATEGDAHTGGGSPASRDLVVSYVNTNMGLKFRQLLWFHISALTFGRETVIEYLVSFGVWIRTPPAYRPPNAPILSTLPETTVV

SEQ ID NO: 2 - protein, 274 aa, synthetic construct
  REGION 1..274: note = Description of sequence: amino acid sequence of shrimp tropomyosin protein
MKLEKDNAMDRADTLEQQNKEANNRAEKSEEEVHNLQKRMQQLENDLDQVQESLLKANNQLVEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 3 - protein, 70 aa, synthetic construct
  REGION 1..70: note = Description of sequence: amino acid sequence of P1 epitope
RGVPHIVMVDAYKRYKGGGSMKLEKDNAMDRADTLEQQNKEANNRAEKSEEEVHNLQKRMQQLENDLDQV

SEQ ID NO: 4 - protein, 64 aa, synthetic construct
  REGION 1..64: note = Description of sequence: amino acid sequence of P2 epitope
RGVPHIVMVDAYKRYKGGGSVHNLQKRMQQLENDLDQVQESLLKANNQLVEKDKALSNAEGEVA

SEQ ID NO: 5 - protein, 60 aa, synthetic construct
  REGION 1..60: note = Description of sequence: amino acid sequence of P3 epitope
RGVPHIVMVDAYKRYKGGGSALSNAEGEVAALNRRIQLLEEDLERSEERLNTATTKLAEA

SEQ ID NO: 6 - protein, 68 aa, synthetic construct
  REGION 1..68: note = Description of sequence: amino acid sequence of P4 epitope
RGVPHIVMVDAYKRYKGGGSNTATTKLAEASQAADESERMRKVLENRSLSDEERMDALENQLKEARFL

SEQ ID NO: 7 - protein, 65 aa, synthetic construct
  REGION 1..65: note = Description of sequence: amino acid sequence of P5 epitope
RGVPHIVMVDAYKRYKGGGSNQLKEARFLAEEADRKYDEVARKLAMVEADLERAEERAETGESKI

SEQ ID NO: 8 - protein, 60 aa, synthetic construct
  REGION 1..60: note = Description of sequence: amino acid sequence of P6 epitope
RGVPHIVMVDAYKRYKGGGSERAETGESKIVELEEELRVVGNNLKSLEVSEEKANQREEA

SEQ ID NO: 9 - protein, 64 aa, synthetic construct
  REGION 1..64: note = Description of sequence: amino acid sequence of P7 epitope
RGVPHIVMVDAYKRYKGGGSEKANQREEAYKEQIKTLTNKLKAAEARAEFAERSVQKLQKEVDR

SEQ ID NO: 10 - protein, 63 aa, synthetic construct
  REGION 1..63: note = Description of sequence: amino acid sequence of P8 epitope
RGVPHIVMVDAYKRYKGGGSAERSVQKLQKEVDRLEDELVNEKEKYKSITDELDQTFSELSGY

SEQ ID NO: 11 - protein, 91 aa, synthetic construct
  REGION 1..91: note = Description of sequence: amino acid sequence of exemplary SpyCatcher peptide
SATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 12 - protein, 16 aa, synthetic construct
  REGION 1..16: note = Description of sequence: amino acid sequence of exemplary SpyTag peptide
RGVPHIVMVDAYKRYK

(19) United States
(12) Patent Application Publication
(10) Pub. No.: US 2026/0053900 A1
(43) Pub. Date: Feb. 26, 2026

(54) USE OF VIRUS-LIKE PARTICLES IN VACCINATION

(71) Applicant: The Chinese University of Hong Kong, Shatin (CN)

(72) Inventors: Yee Yan Christine WAI, Kowloon (CN); Ting Fan LEUNG, Shatin (CN); Yat Hin Nicki LEUNG, Kowloon (CN)

(21) Appl. No.: 19/195,982

(22) Filed: May 1, 2025

Related U.S. Application Data
(60) Provisional application No. 63/642,559, filed on May 3, 2024.

(51) Int. Cl. A61K 39/00; A61K 9/00; A61K 39/395; A61P 37/08; C07K 14/005; C07K 14/435

(52) U.S. Cl. CPC A61K 39/0003; A61K 9/0019; A61K 39/001; A61K 39/395; A61P 37/08; C07K 14/005; C07K 14/43504; A61K 2039/5258; A61K 2039/54; A61K 2039/545; A61K 2039/6075; C07K 2319/70; C12N 2730/10122; C12N 2730/10123; C12N 2730/10134; C12N 2730/10151

(57) ABSTRACT

This invention provides a virus-like particle (VLP) that is formed by a protein of a viral origin (such as from a Hepatitis B virus) and derivatized by way of an isopeptide bond formed between two partner peptides (such as a SpyCatcher/SpyTag connection) to present on the VLP surface one or more antigenic epitopes of a target antigen. Also provided are methods of making the VLP, compositions comprising the VLP, as well as applications of the VLP and the compositions for modulating a recipient's immune response to the target antigen, including immunization against the antigen or desensitization to the antigen.

### RELATED APPLICATIONS

This application claims priority to U.S. Provisional Patent Application No. 63/642,559, filed May 3, 2024, the contents of which are hereby incorporated by reference in the entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 11, 2025, is entitled “080015-1507146-040210US_ST26.xml”, and is 17,542 bytes in size.

### BACKGROUND OF THE INVENTION

Allergies are a collection of diseases caused by hypersensitivity of a person's immune system to typically harmless substances in the environment. Common allergens include pollen and certain foods. In addition, insect stings and medications also frequently cause severe allergic reactions. Allergies are common, especially in the developed world, where about 20% of people are affected by allergic rhinitis, up to 18% of people have asthma, and about 20% have or have had atopic dermatitis at some point in their life. Food allergy, another highly prevalent allergic disorder, is now a global health concern affecting up to 10% of the general population.

Allergen-specific immunotherapy (AIT) is the only curative treatment for allergies. Conventional AIT protocols involve a prolonged procedure, by which incrementally increased doses of the inciting allergen are given for years for immune modulation. The AIT protocols currently in use have significant efficacy limitations to develop sustained unresponsiveness in recipients to the allergen, however, not to mention limitations in long-term remission of the disease. The associated side effects and long treatment course also hinder patient adherence and increase the cost of therapy. Thus, there exists an urgent need to develop alternative approaches to mitigate side effects and enhance efficacy for AIT in the treatment of food allergy. This invention fulfills this and other related needs.

### BRIEF SUMMARY OF THE INVENTION

The present invention provides a novel virus-like particle (VLP) formed by a modified Hepatitis B virus (HBV) core capsid protein (HBcAg) that is capable of presenting epitopes of a target antigen and is therefore useful for modulating the immune response to the target antigen in a recipient, either to promote B cell-based humoral response and/or T cell-based cytotoxic response specifically directed to the target antigen or to achieve desensitization or tolerance of the target antigen. Thus, the first aspect of the present invention relates to a method for making an antigen epitope-presenting virus-like particle (VLP). The method includes the first step of forming a VLP using a modified viral structural protein that comprises at least a portion of the viral protein fused to a first heterologous peptide (such as a SpyCatcher peptide), and then the second step of reacting the VLP with a fusion antigenic peptide that comprises an epitope of a target antigen fused with a second heterologous peptide (such as a SpyTag peptide) so that an isopeptide bond forms between the first and second heterologous peptides in order to yield a VLP presenting the epitope of the target antigen. The nature of the first and second heterologous peptides is such that they act as partners to form an isopeptide bond: one of the peptides has a non-α-amine or carboxy group (e.g., via the side chain non-α-amine group of a lysine residue within the peptide) to form an isopeptide bond with the other peptide at its α- or non-α-carboxy or amine group (e.g., via the non-α-carboxy group of an aspartate residue within the peptide), respectively. For example, the claimed method includes the first step of forming a VLP with a modified viral structural protein comprising at least a portion of the viral protein fused to a SpyCatcher peptide, and then the second step of reacting the VLP with a fusion antigenic peptide comprising an epitope of a target antigen fused with a SpyTag peptide to yield a VLP presenting the epitope of the target antigen. Alternatively, the claimed method includes the first step of forming a VLP with a modified viral structural protein comprising at least a portion of the viral protein fused to a Spy Tag peptide, and then the second step of reacting the VLP with a fusion antigenic peptide comprising an epitope of a target antigen fused with a SpyCatcher peptide to yield a VLP presenting the epitope of the target antigen.

In some embodiments, the viral structural protein for use in the method is a Hepatitis B virus core protein (HBcAg) or a truncated version of the protein, for example, having the amino acid sequence set forth in SEQ ID NO:1. In some embodiments, the target antigen is a shrimp tropomyosin, for example, having the amino acid sequence set forth in SEQ ID NO:2. In some embodiments, the epitope of the target antigen is any one of P1 to P8, having the amino acid sequence set forth in SEQ ID NOs: 3-10, respectively. In some embodiments, the method comprises reacting the VLP, which is formed by a modified viral protein fused to a first partner peptide capable of forming an isopeptide bond (e.g., a SpyCatcher peptide), with one, two, or more fusion antigenic peptides, each comprising a different epitope fused with a second partner peptide capable of forming an isopeptide bond (e.g., a SpyTag peptide). In some embodiments, the modified viral protein is a dimer of a truncated version of HBcAg having a first partner peptide capable of forming an isopeptide bond (e.g., a SpyCatcher peptide) inserted in the MIR region of the HBcAg. In some embodiments, the method comprises reacting the VLP, which is formed by a modified viral protein fused to a first partner peptide capable of forming an isopeptide bond (e.g., a SpyTag peptide), with one, two, or more fusion antigenic peptides, each comprising a different epitope fused with a second partner peptide capable of forming an isopeptide bond (e.g., a SpyCatcher peptide). In some embodiments, the modified viral protein is a dimer of a truncated version of HBcAg having a SpyTag peptide inserted in the MIR region of the HBcAg. In some embodiments, the molar ratio between the modified viral protein and each of the fusion antigenic peptides is about 1 to 5, about 1 to 10, about 1 to 20, or up to about 1 to 50. In some embodiments, the two or more fusion antigenic peptides each comprise a different epitope independently selected from P1 to P8.

In a second aspect, the present invention provides an antigen epitope-presenting VLP made by the method described above and herein. Also provided is a comprising the VLP and a pharmaceutically or physiologically acceptable excipient. In some embodiments, the VLP composition further comprising an adjuvant. In some embodiments, the VLP composition is formulated for administration by injection, such as subcutaneous or intramuscular injection. In some embodiments, the VLP composition is formulated for mucosal delivery, for example, by oral ingestion or nasal inhalation. In a related aspect, the present invention provides a kit comprising the VLP composition described above and herein. In some embodiments, the VLP composition in the kit is a vaccine against a shellfish allergy. In some embodiments, the vaccine is adapted for administration by injection (e.g., subcutaneous or intramuscular injection) or for administration by oral or nasal delivery by means of ingestion or inhalation, respectively.

In a third aspect, the present invention provides a method for eliciting an immune response in an individual by administering to the individual an effective amount of the VLP composition described above and herein. In some embodiments, the administering step comprises injection of the VLP composition, for example, by subcutaneous injection, or by oral ingestion or by nasal inhalation. In some embodiments, the immune response comprises a B cell-based humoral response and/or T cell-based cytotoxic response against the target antigen. In other cases, the immune response is tolerance or desensitization for the target antigen. In some embodiments, the method includes repeated administration step by at least once. In the case where the method is used to induce immunotolerance for the target antigen, the method often further comprises, prior to the administering step, a pre-treatment step of contacting the recipient with the target antigen to sensitize the recipient to the antigen. In such cases, the administering step may be repeated at least twice at a weekly or bi-weekly interval following the pre-treatment step. In some embodiments, the administration step is repeated at least four to ten or more times at a weekly or bi-weekly interval following the pre-treatment step. In some embodiments, the individual is previously diagnosed or is suspected to suffer from an allergy to the target antigen. In some embodiments, the individual is previously diagnosed or is suspected to suffer from a shellfish allergy, and wherein the individual is first contacted with a shrimp tropomyosin prior to being administered a composition comprising VLPs produced from the method of this invention and presenting one or more shrimp tropomyosin epitopes such as one or more of P1 to P8.

### BRIEF DESCRIPTION OF THE DRAWINGS

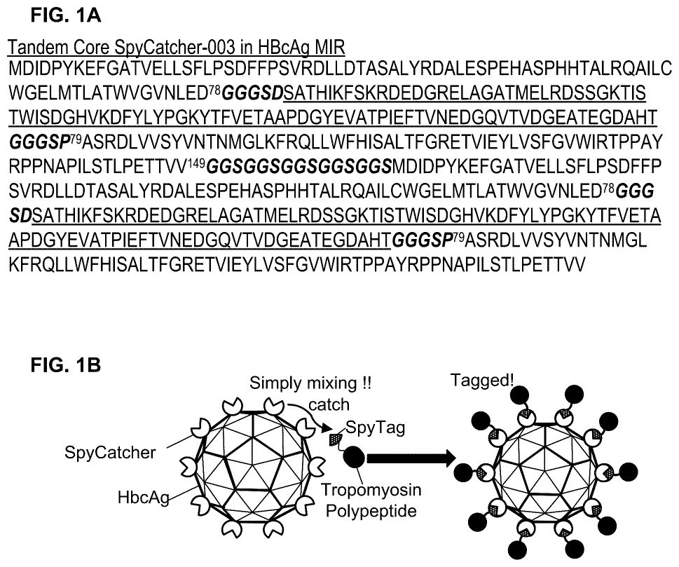

Design of HBcAg-SpyCatcher and SpyTag vaccine. FIG. 1A: Nucleotide sequence of the tandem core HBcAg with SpyCatcher sequence (underlined) inserted in MIR region for surface display. Alanine substitution at C48 and C107 (boxed) are introduced to increase solubility of HBcAg. Tandem fusion of two HBcAg open reading frames by linker sequences (bold and italic) to overcome steric clashes and allow correct assembly. FIG. 1B: Illustration of the plug-and-display system based on the SpyTag/SpyCatcher technology. HBcAg-polypeptide vaccine is constructed by simple mixing of the two molecules.

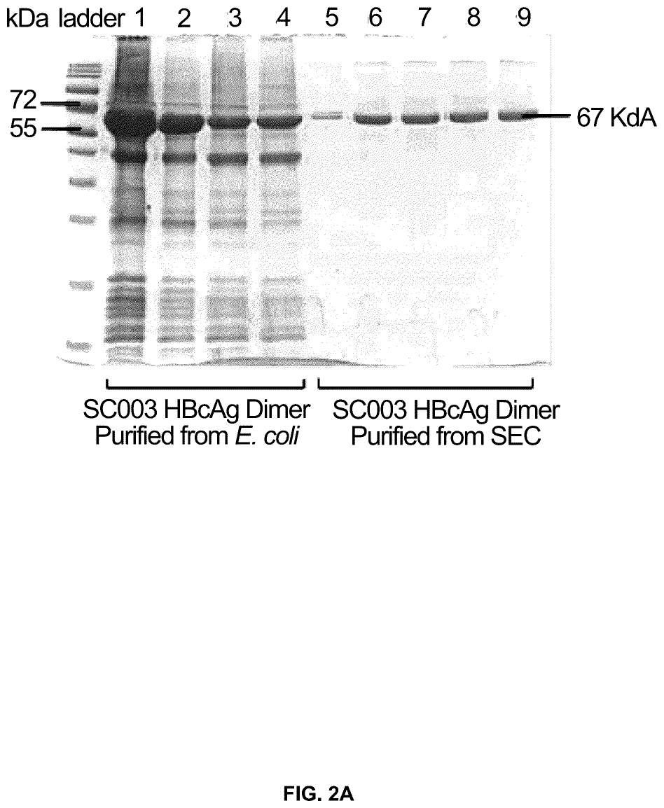

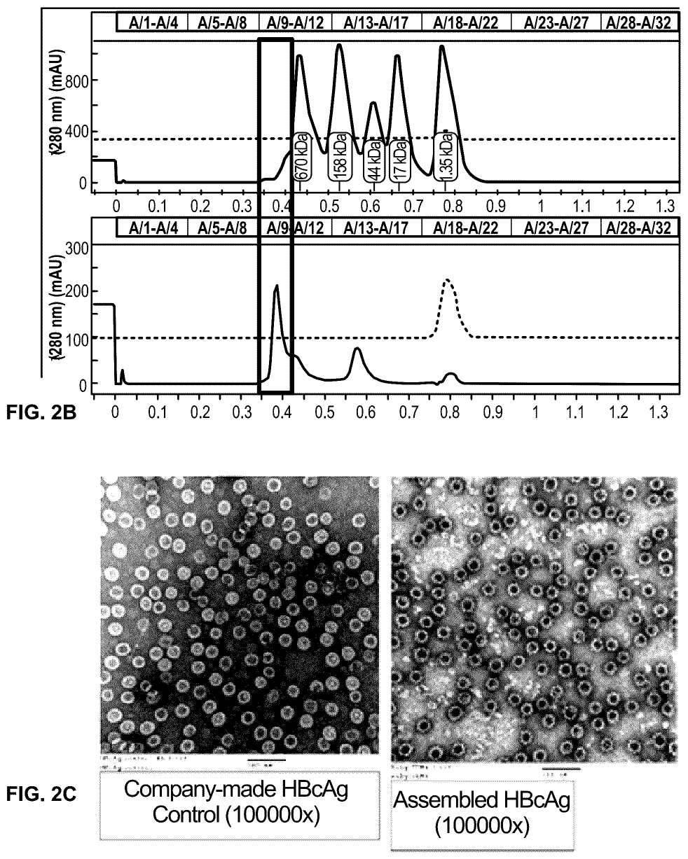

HBcAg production. FIG. 2A: SDS-PAGE showing the in-house purified HBcAg with SpyCatcher (67 kDa protein). FIG. 2B: Chromatogram showing the correct size and so assembly of HBcAg with size >670 kDa (lower graph) when comparing to the calibration standard (upper graph, boxed). FIG. 2C: Transmission electron microscope (TEM) images showing the correct assembly of our in-house purified HBcAg comparable to commercial HBcAg control.

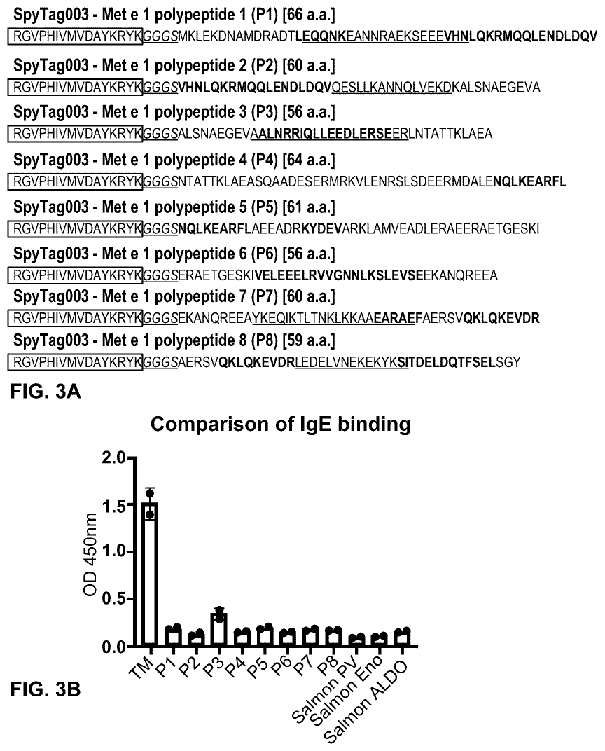

Design and characterization of tropomyosin polypeptides. FIG. 3A: Amino acid sequences of the eight designer polypeptides of tropomyosin (TM). SpyTag sequence (boxed) is introduced at the N-terminal. Each polypeptide contains at least one IgE-binding epitope (bold) and T cell epitopes (underlined). Linker sequences (italic and double underlined) were introduced between SpyTag and TM peptide. Length of each polypeptide is also noted. FIG. 3B: Comparison of IgE-binding of polypeptides with tropomyosin (TM). Note that the IgE binding activity of the polypeptides are as low as the irrelevant allergens including parvalbumin (PV), enolase (Eno) and aldolase (ALDO) from salmon.

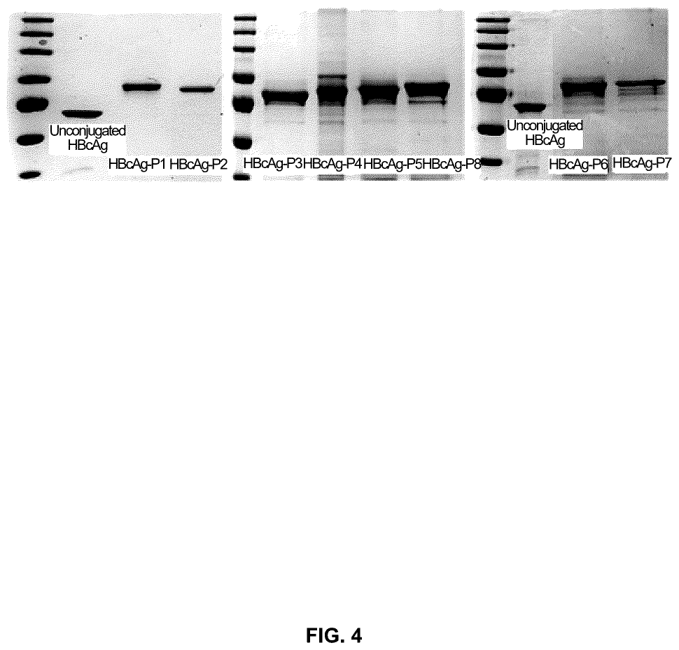

HBcAg-polypetide vaccine production. FIG. 4: SDS-PAGE showing the efficient conjugation of polypeptides on HBcAg, as noted by the increase in protein size of the conjugated molecule compared to unconjugated HBcAg.

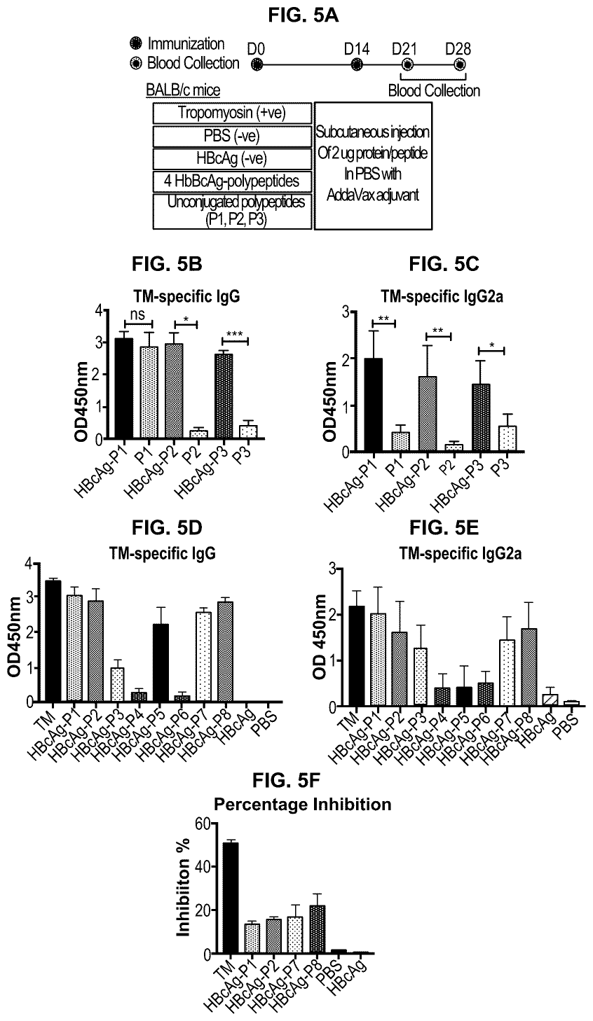

Immunogenicity of HBcAg-polypeptide vaccines. FIG. 5A: Regimen of mouse immunization. Blood was collected from immunized animals and analyzed on ELISA for TM-specific IgG and IgG2a. FIG. 5B: TM-specific IgG levels of mice immunized with P1, P2 or P3 with/without conjugation to HBcAg. Note that conjugation to HBcAg efficiently increased the immunogenicity of P2 and P3. FIG. 5C: TM-specific IgG2a levels of mice immunized with PI, P2 or P3 with/without conjugation to HBcAg. Note that conjugation to HBcAg efficiently increased the induction of allergen-specific IgG2a antibody. FIG. 5D: Comparison of TM-specific IgG titers in mice immunized with different vaccines. Note that HBcAg-P1, P2, P5, P7 and P8 induced high levels of TM-specific IgG. FIG. 5E: Comparison of TM-specific IgG2a titers in mice immunized with different vaccines. Note that HBcAg-P1, P2, P7 and P8 induced higher IgGa titers. Overall, HBcAg promoted the immunogenicity of polypeptide and specifically induced IgG2a. HBcAg-P1, -P2, -P7 and-P8 showed prominent induction of IgG and IgG2a similar to tropomyosin (TM). FIG. 5F: IgG2a induced by these four vaccines inhibited 13.6%-21.8% of IgE binding (sera of shrimp allergic subjects).

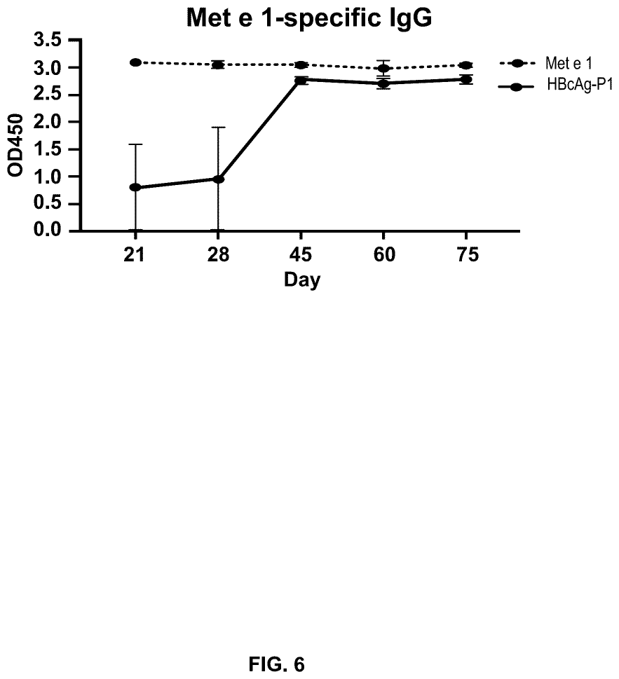

Durability of antibody response induced by HBCAg-polypeptide vaccine. FIG. 6: Blood was collected on day 21, 28, 45, 60 and 75 from BALB/c mice immunized with HBcAg-P1 or Met e 1 to monitor antibody response. Note that the antibody response induced by the vaccine was sustained at day 75 (60 days post-vaccination) that highlight the strong immunogenicity of the vaccine.

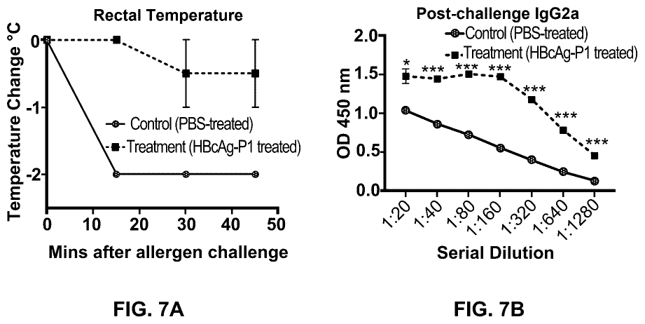

Therapeutic potential of HBcAg-polypeptide vaccines. FIG. 7A: treatment with HBcAg-P1 protected against the drop in rectal temperature. FIG. 7B: treatment remarkably induced IgG2a to block IgE to protect against shrimp allergy (*P<. 05; *** P<. 0001).

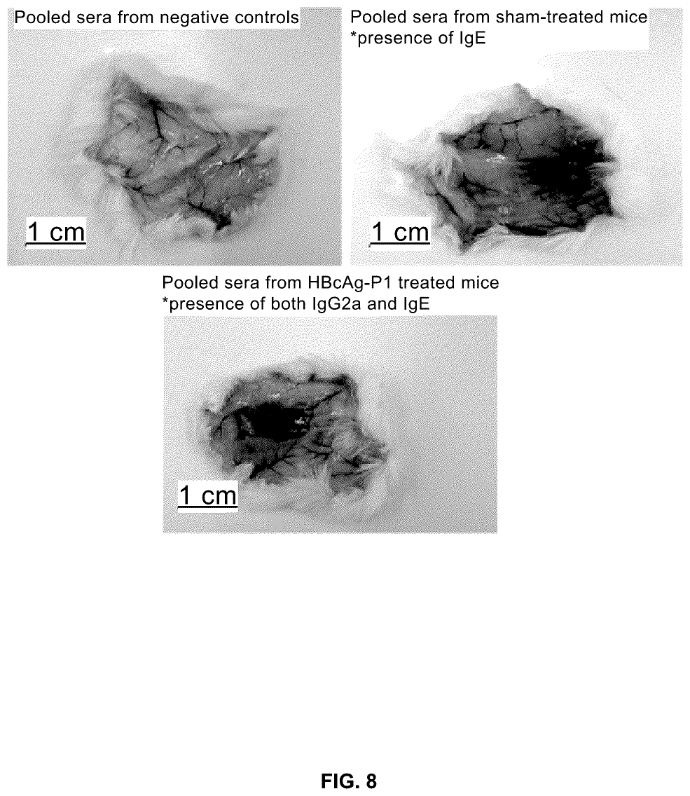

Inhibitory potential of the vaccine-induced IgG. FIG. 8: Passive cutaneous anaphylaxis (PCA) experiment was conducted by intravenously injecting sera from negative control mice (no sensitization/treatment), sham-treated mice (sensitized by shrimp extract and sham-treated with PBS only) and HBCAg-P1 treated mice to naïve BALB/c mice. This was followed by the intradermal injection of tropomyosin with Evan's blue dye at the back skin. Noted the reduced leakage of Evan's blue dye with sera from vaccine-treated mice, which indicated the reduced degranulation due to the specific inhibition of IgE binding with vaccine-induced IgG antibodies.

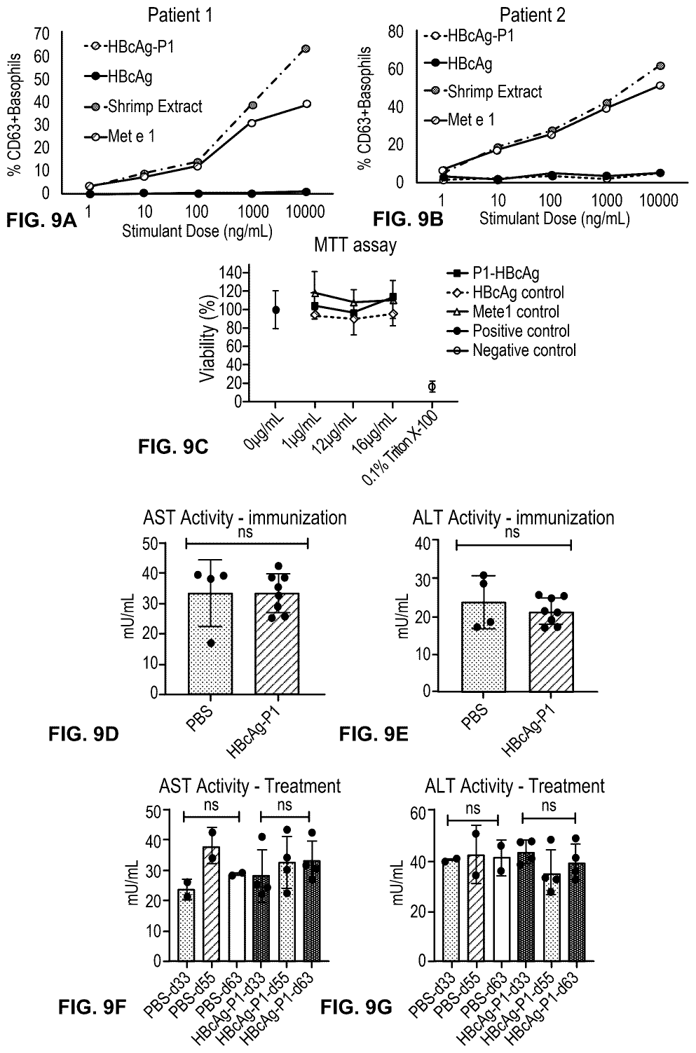

Safety of the HBcAg-P1 vaccine. FIG. 9A and FIG. 9B illustrate that the HBcAg-P1 vaccine and HBcAg carrier did not induce basophil activation (i.e. % of CD63+basophil cells) in shrimp-allergic patients but only Met e 1. FIG. 9C MTT assay using PBMCs from healthy donor with data presented as % viability of cells, which noted that the vaccine and HBcAg carrier did not trigger cell death. In vivo toxicity of the vaccine in mice was also measured, by which mice immunized with HBcAg-P1 had similar activity of FIG. 9D aspartate aminotransferase (AST) and FIG. 9E alanine transferase (ALT) with control mice sham-immunized with PBS. FIG. 9F. and FIG. 9G Both AST and ALT activities remained the same along the experiment in mice with shrimp allergy and treated with HBcAg-P1. These indicate no hepatotoxicity induced by the vaccine and so the safety of the vaccine.

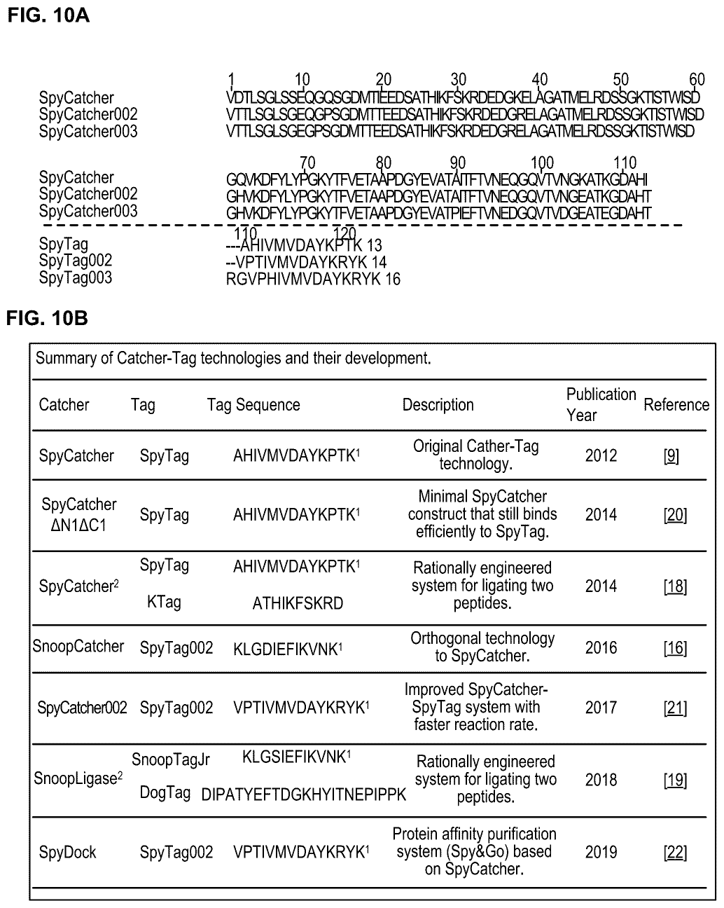

Exemplary peptide pairs capable of forming isopeptide bonds in the Plug-and-Display scheme. FIG. 10A: Exemplary SpyCatcher and SpyTag sequences. FIG. 10B: list of known peptide pairs in the Plug-and-Display scheme.

### Definitions

“Hepatitis B virus” or “HBV” refers to a partially double-stranded DNA virus, a species of the genus Orthohepadnavirus and a member of the Hepadnaviridae family of viruses, which causes infectious hepatitis transmitted by exposure to bodily fluids of an infected and is distinguished from Hepatitis A virus (HAV), Hepatitis C virus (HCV), Hepatitis D virus (HDV), or Hepatitis E virus (HEV) in terms of serological characteristics as well as genomic sequence. “Hepatitis B core antigen (HBcAg)” or “HBV capsid protein” is the main structural protein of HBV icosahedral nucleocapsid and plays a significant role in the replication of the virus.

As used herein, the term “virus-like particle” or “VLP” refers to a structure that in at least one attribute resembles a virus but is not infectious due to the lack of a viral genome. “VLP” refers to a nonreplicating viral shell, e.g., derived from hepatitis B virus proteins such as a core capsid protein. VLPs are generally composed of one or more viral proteins, including, but are not limited to the structural proteins referred to as capsid proteins or core proteins, or their modified variants including those with less than full-length of the corresponding wild-type protein and/or with one or more amino acid modification (e.g., insertion, deletion, or substitution) yet retaining the capability to form VLPs. VLPs can form spontaneously upon recombinant expression of the protein or its variant in an appropriate expression system.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a variant HBcAg protein useful for the method of this invention has at least 80% sequence identity, preferably 85%, 90%, 91%, 92%, 93, 94%, 95%, 96%, 97%, 98%, 99%, or 100% identity, to a reference sequence, e.g., an exemplary truncated HBcAg protein having the amino acid sequence of SEQ ID NO: 1, permitting modification in the form of deletion, insertion or substitution of one or more amino acid residues, such as a double mutant harboring C48A and C107A substitutions), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75 to 100 or 200 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous nucleic acid,” as used herein, refers to a nucleic acid not endogenous to a specified virus (such as the hepatitis B virus), i.e., from a source other than that virus species. The term “heterologous polypeptide,” as used herein, refers to a peptide or polypeptide not endogenous to a specified virus (such as the hepatitis B virus), or a peptide or polypeptide to a protein or peptide coded for by a DNA sequence that is not endogenous to the native genome of that virus, i.e., from a source or organism other than HBV. When the word “heterologous” is used to describe two components in a recombinant construct, such as a fusion protein or a polynucleotide sequence, it refers to the fact that they are derived from two separate and unrelated origins and are not found together in the same fashion in nature.

As used herein, two “partner peptides” capable of forming an isopeptide bond are two peptides that one of the peptides offers a non-α-amine (—NH₂) or carboxy (—COOH) group, such as the amine group of a lysine residue or the carboxy side group of an aspartate residue, to react with an α- or non-α-carboxy or amine group, respectively, in the other peptide. This scheme of isopeptide bond-forming between a pair of partner peptides, termed “Tag” and “Catcher,” is also known as the “plug-and-display” system and include several known sets of such partner peptides, including but are not limited to those shown in FIG. 10. It is understood that these partner peptides may have a degree of sequence variability from the exemplary sequences shown in this disclosure. For example, the amino acid sequence for the SpyCatcher or SpyTag peptide may have at least 80%, 85%, 90%, or 95% sequence identity to SEQ ID NO: 11 or 12, respectively.

As used in this application, an “increase” or a “decrease” refers to a detectable positive or negative change in quantity from a predetermined comparison value, e.g., an established standard control or an average value prior to or in the absence of an event (such as an average level of IgE in a subject upon exposure to an allergen without treatment). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the control value, typically within +10% of the standard control, or within +5%, 2%, or even less variation from the control value.

The term “effective amount” as used herein refers to an amount of a given substance that is sufficient in quantity to produce a desired effect. For example, an effective amount of a VLP displaying epitope(s) of a target antigen is the amount of said VLP to achieve in the recipient a decreased level of IgE against the target antigen, such that the symptoms of an allergy to the target antigen are reduced, reversed, eliminated, prevented, or delayed of the onset in a patient who has been given the polynucleotide for therapeutic purposes. An amount adequate to accomplish this is defined as the “therapeutically effective dose.” The dosing range varies with the nature of the therapeutic agent being administered and other factors such as the route of administration and the severity of a patient's condition.

A “pharmaceutically acceptable” or “physiologically acceptable” material is one that is not biologically harmful or otherwise undesirable, i.e., the material may be administered to an individual along with the HBcAg VLPs or the compositions of the present invention without causing any undesirable biological effects. Neither would the material interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “adjuvant” refers to a compound that, when administered in conjunction with an antigen, augments the immune response to the antigen, but does not generate an immune response to the antigen when administered alone. Adjuvants can augment an immune response by several mechanism including lymphocyte recruitment, stimulation of B and/or T cells, and stimulation of macrophages.

An “immune response” to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+T-cells. Hence, an immunological response may include one or more of the following effects: the production of antibodies by B-cells; and/or the activation of suppressor T-cells and/or γΔ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immunotolerance,” “immune tolerance,” or simply “tolerance” refers to a state of unresponsiveness of the immune system to substances that would otherwise have the capacity to elicit an immune response in a host or recipient. Immunotolerance is induced by prior exposure to the specific antigen and contrasts with conventional immune-mediated elimination of foreign antigens (see “immune response” in the last section). Tolerance is classified into central tolerance or peripheral tolerance, depending on where the state is originally induced—in the thymus and bone marrow (central) or in other tissues and lymph nodes (peripheral). Central tolerance is the main way the immune system learns to discriminate self from non-self. Peripheral tolerance is key to preventing over-reactivity of the immune system to various environmental entities (allergens, gut microbes, etc.).

As used herein, the term “host” or “recipient” refers to an individual who has been administered a substance of interest, e.g., the HBcAg VLPs or the composition of the present invention. It may encompass humans of any age and either gender as well as other animals.

The term “mucosal delivery” relates to delivery of a composition to and be absorbed through a mucous membrane, such as the mucosa of the gastro-intestinal tract (e.g., the buccal or labial mucosa), the mucosa of the mouth or the eyes, or the mucosa of the respiratory tract (e.g., the nasal mucosa).

As used herein, the term “about” denotes a range of +/−10% of a stated value. For instance, “about 10” denotes a range of 9-11 (10+/−1).

### DETAILED DESCRIPTION OF THE INVENTION

### I. INTRODUCTION

There have been previous efforts to use various forms of virus-like particles (VLPs) as vehicles to deliver therapeutic agents or vaccines, see, e.g., U.S. Pat. Nos. 8,906,862; 10,053,494; and 11,466,055. Taking advantage of the “plug-and-display” methodology, the present invention relates to a novel VLP constructed from a viral structural protein, such as Hepatitis B virus core protein (HBcAg), fused with a first peptide sequence of a “plug-and-display” pair (such as a SpyCatcher sequence) for the convenient surface display of any proteins or peptides fused with a second peptide sequence of the “plug-and-display” pair (such as a SpyTag sequence) for tailored AIT vaccine to achieve precision treatment of allergies (such as food allergy). HBcAg is a nano-carrier inducing strong humoral and cellular immune responses. With the successful production of this HBcAg-SpyCatcher, HBc-Ag-polypeptide vaccines for shrimp allergy were constructed for the first time. The vaccines were shown to be able to induce blocking IgG antibodies and protect against shrimp allergy. This plug-and-display HBcAg system is the first-ever successful prototype of personalized vaccine for effective treatment of allergies including food allergies.

### II. Production of Polypeptides

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The genomic sequence of a viral structural protein, such as a human Hepatitis B virus core protein (HBcAg), a truncated variant HBcAg 149 having the amino acid sequence of SEQ ID NO: 1, a polynucleotide encoding a polypeptide having the amino acid sequence SEQ ID NO: 1 or its variants/mutants, and synthetic oligonucleotides can be verified after cloning or subcloning using, e.g., the chain termination method for sequencing double-stranded templates of Wallace et al., Gene 16:21-26 (1981).

### B. Chemical Synthesis of Peptides

The amino acid sequence of target antigen protein such as shrimp tropomyosin and its nucleotide coding sequence are known and provided herein. A polypeptide comprising the full-length tropomyosin protein or its epitopes thereof (e.g., P1-P8) including and one or more “plug-and-display” peptide sequence(s) (e.g., SpyCatcher or SpyTag sequence) thus can be chemically synthesized using conventional peptide synthesis or other protocols well known in the art, see, e.g., Wai et al., (2014) PloS ONE 9 (11): e111649. doi: 10.1371/journal.pone.0111649.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N. Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl) phenoxy resin; tert-alkyloxycarbonyl-hydrazidated resins, and the like. Such resins are commercially available and their methods of preparation are known by those of ordinary skill in the art. Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### C. Recombinant Expression of Polypeptides

A HBcAg protein of SEQ ID NO: 1, its variant/mutant (e.g., having at least 90 or 95% sequence identity to SEQ ID NO:1), or any fusion polypeptide comprising a wild-type HBcAg protein or its variant/mutant and one partner peptide of the “plug-and-display” system (such as a SpyCatcher peptide sequence) can be produced using routine techniques in the field of recombinant genetics, relying on the polynucleotide sequences encoding the polypeptide disclosed herein.

To obtain high level expression of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. One exemplary eukaryotic expression vector is an adenoviral vector, an adeno-associated vector, or a retroviral vector.

Standard transfection methods can be used to produce bacterial, mammalian, yeast, insect, or plant cell lines that express large quantities of a recombinant polypeptide (e.g., a HBcAg-SpyCatcher polypeptide), which is then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

When a recombinant polypeptide, e.g., a HBcAg-SpyCatcher polypeptide, is expressed in host cells in satisfying quantity, its purification can follow the standard protein purification procedure including Solubility fractionation, size differential filtration, and column chromatography. These standard purification procedures are also suitable for purifying HBcAg-SpyCatcher polypeptides comprising a HBcAg sequence (wild-type or variant) obtained from chemical synthesis. The identity of the HBcAg protein may be further verified by methods such as immunoassays (e.g., Western blot or ELISA) and mass spectrometry.

### III. Construction of Virus-Like Particles

Virus-like particles (VLPs) are diverse nanoparticles (sized 20-200 nm) that are formed by structural viral proteins, such as capsids, and can self-assemble. They resemble viruses and generally take the form of original virus source, e.g., icosahedral, rod shaped, or globular. VLPs are, however, noninfectious because they lack viral genetic material. As VLPs can mimic the three dimensional conformation of the native viruses from which they were derived and display high densities of repetitive effective antigenic epitopes on their surface, can be used as delivery vehicles such as vaccines. Their utility as vaccines is further enhanced by their ability to readily drain to lymph nodes and interact with antigen-presenting cells and B cells (see, e.g., Mohsen et al., Vaccines. 2018; 6:37). This feature mediates effective stimulation of B and T-cell responses.

The functionality of VLPs can be further increased through modifying their exterior or interior surface to display heterologous epitopes of interest using different methods like peptide conjugation, genetic fusion, and chemical crosslinking (see, e.g., Schwarz K., et al. Eur. J. Immunol. 2005; 35:816-821; Brune K. D., et al. Sci. Rep. 2016; 6:19234).

VLPs can be differentiated based on their structural complexity. Capsid proteins can be arranged in one, two or three layers. The layers can contain more than one structural protein. Further some VLPs such as those derived from HIV-1 and influenza virus, have a lipid layer, termed a lipid envelope, that contains viral surface antigens surrounding the capsid structure. Frequently enveloped VLPs contain a matrix protein located immediately inside the host-derived lipid membrane in which the viral glycoproteins are embedded.

VLPs have been synthesized using a wide range of expression systems bacteria, yeast, plant cells, insect cells and mammalian cell lines. Eukaryotic expression systems are generally more suitable for the production VLPs since these expression systems are capable of at least two functions not found in bacterial expression systems: post-translational modifications and the ability to produce enveloped VLPs.

One aspect of the invention relates to construction of a viral structural protein, e.g., HBV core protein (HBcAg), for self-assembly into virus-like particles (VLPs). Various constructs of similar viral capsid or core proteins can be used for formation of VLPs (see, e.g., Expression and self-assembly of empty virus-like particles of hepatitis E virus. Li T C, Yamakawa Y, Suzuki K, Tatsumi M, Razak M A, Uchida T, Takeda N, Miyamura T., J Virol. 1997 October; 71 (10): 7207-13. Essential elements of the capsid protein for self-assembly into empty virus-like particles of hepatitis E virus. Li T C, Takeda N, Miyamura T, Matsuura Y, Wang J C, Engvall H, Hammar L, Xing L, Cheng R H. J Virol. 2005 October; 79 (20): 12999-3006.).

As described in the Examples of this application, a truncated HBV core protein having the amino acid sequence of SEQ ID NO: 1 can be used as a construct for formation of VLPs in vitro. Preferably, an HBV core protein (including a truncated version, optionally containing modification to one or more amino acid residues) is recombinantly produced (e.g., in a dimer form) with a partner peptide of the “plug-and-display” system (e.g., a SpyCatcher peptide) inserted into the major immunodominant region (MIR) of the protein as the base construct for the assembly of VLPs in vitro. The presence of the peptide of the “plug-and-display” system, such as the SpyCatcher peptide, on the surface of the VLPs allows for the subsequent conjugation onto the VLP surface of fusion peptides comprising an antigenic epitope fused with a complementary partner peptide of the “plug-and-display” system, such as the Spy Tag peptide, such that the epitope will ultimately be displayed on the VLP surface. Conversely, the HBV core protein or its variant such as a truncated form and/or with further point mutation(s) may be fused in the same fashion with a SpyTag peptide, such that the subsequent conjugation reaction will take place between the VLPs and fusion peptides comprising an antigenic epitope fused with a SpyCatcher peptide. In all of these fusion polypeptides intended for VLP formation and the fusion peptides intended to graft antigenic epitope(s) onto the VLP surface, one or more peptide linkers may be used at either or both C-terminus and N-terminus as well as between any two of the fusion partners so as to ensure their being joined in a functional manner.

### IV. Epitope Conjugation and Purification of Vlps

One aspect of the invention relates to methods for production and purification of virus-like particles (See, Expression and self-assembly of empty virus-like particles of hepatitis E virus. Li T C, Yamakawa Y, Suzuki K, Tatsumi M, Razak M A, Uchida T, Takeda N, Miyamura T., J Virol. 1997 October; 71 (10): 7207-13. Essential elements of the capsid protein for self-assembly into empty virus-like particles of hepatitis E virus. Li T C, Takeda N, Miyamura T, Matsuura Y, Wang J C, Engvall H, Hammar L, Xing L, Cheng R H. J Virol. 2005 October; 79 (20): 12999-3006. Niikura M et al, Chimeric recombinant hepatitis E virus-like particles as an oral vaccine vehicle presenting foreign epitopes. Virology 2002; 293:273-280). As noted in an earlier section, various expression systems can be used to express the modified viral structural protein (e.g., HBcAg-SpyCatcher fusion polypeptide) of the present invention. Examples of expression systems useful for the production of virus-like particles of the present invention include, but are not limited to, bacterial expression system (e.g., Escherichia coli), insect cells, yeast cells and mammalian cells. Preferred expression system of the present invention includes baculovirus expression systems using insect cells. General methods, for example, for handling and preparing baculovirus vectors and baculoviral DNA, as well as insect cell culture procedures, are outlined in A Manual of Methods for Baculovirus Vectors and Insect Cell Culture Procedures.

Once recombinantly expressed, a modified viral structural protein (e.g., HBcAg protein having the amino acid sequence set forth in SEQ ID NO: 1 fused at its N-terminus with a SpyCatcher peptide having the amino acid sequence set forth in SEQ ID NO:11) can spontaneously form a virus-like particle (VLP) in a structure similar to the intact virus but without the genetic material of the virus and therefore incapable of replication. Because of the presence of the SpyCatcher peptide on the VLP surface, SpyTag-labelled peptides representing one or more distinct epitopes of a pre-determined target antigen can be conjugated to the VLP by way of the SpyCatcher/SpyTag connection.

The reaction of the partner peptides of the “plug-and-display” system, such as the SpyCatcher and SpyTag peptides, to form a covalent isopeptide bond between the peptide pair is a well-established technique. The reaction conditions are described herein and can be found in user instructions provided by commercial suppliers of SpyCatcher and Spy Tag products (e.g., Bio-Rad Laboratories, Inc.) as well as in the scientific literature, see, e.g., Zakeri et al., Proc Natl Acad Sci USA 109, E690-697, doi.org/10.1073/pnas. 1115485109 (2012); Li et al., J Mol Biol 426, 309-317, doi.org/10.1016/j.jmb.2013.10.021 (2014); and Fierle et al., Sci Rep 9, 12815 doi.org/10.1038/s41598-019-49233-7 (2019).

To ensure optimal display antigenic epitopes on the VLP surface, the molar ratio of a modified viral structural protein fused with a partner peptide of the “plug-and-display” system such as SpyCatcher (or SpyTag) peptide (e.g., HBcAg-SpyCatcher) and a Spy Tag (or SpyCatcher)-labelled epitope peptide (e.g., P1-SpyTag) can be adjusted from 1:5, 1:10, 1:20, 1:50, or up to 1:100, depending on the nature of the epitope and/or the presence of other partner peptides of the “plug-and-display” system, such as SpyTag (or SpyCatcher)-labelled epitope peptides. In some cases, a mixture of different peptides harboring distinct epitopes of the same or different antigens may be used to react with the modified viral structural protein. Upon completion of the isopeptide bond formation (e.g., SpyCatcher-SpyTag reaction), the VLP displays on its surface one or more epitopes of the target antigen or antigens.

Purification of the virus-like particles of the present invention can be carried out according to the standard technique in the art (See, Li T C, et al., J Virol. 1997 October; 71 (10): 7207-13. Li T C, et al., J Virol. 2005 October; 79 (20): 12999-3006. Niikura M et al., Virology 2002; 293:273-280). The purified VLPs are then resuspended in a suitable buffer.

### V. Pharmaceutical Compositions and Administration

The present invention also provides pharmaceutical compositions or physiological compositions comprising a VLP formed by a modified viral structural protein according to the method of the present invention. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery. See Langer, Science 249:1527-1533 (1990).

The compositions of the present invention can be administered to a host with an excipient. Excipients useful for the present invention include, but are not limited to, vehicles, binders, disintegrants, fillers (diluents), lubricants, glidants (flow enhancers), compression aids, colors, sweeteners, preservatives, suspending/dispersing agents, film formers/coatings, flavors and printing inks.

Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS 21, QS 18, CRL1005, Aluminum salts, MF 59, and Virosomal adjuvant technology. The adjuvants can also comprise a mixture of these substances.

One advantage of the compositions and methods of the present invention is that the compositions of the novel form of antigen-presenting VLPs can be administered to stimulate immune response without an adjuvant. Therefore, the compositions of the present invention are in some cases administered to a host or recipient without any adjuvant.

Another advantage of the present invention is that the compositions of the present invention are suitable for mucosal delivery (e.g., oral or nasal delivery). Because mucosal surfaces are inter-connected, stimulation of one mucosal surface by an antigen can induce mucosal immunity not only on the directly stimulated surface, but also on the distant ones. For example, oral delivery of the compositions of the present invention can protect against respiratory and genital-urinary infections. The compositions of the present invention are also suitable for mucosal delivery by way of the respiratory or digestive tract, such as delivery to the buccal or labial mucosa or the respiratory tract mucosa, including the nasal mucosa.

The pharmaceutical compositions of the present invention can be administered by various routes, e.g., oral, nasal, subcutaneous, transdermal, intramuscular, intravenous, or intraperitoneal. The one preferred route of administering the pharmaceutical compositions is oral delivery, whereas another preferred route of administration is via injection, e.g., subcutaneous injection. Typically, the administration is carried out at a dose of about 0.1-100 μg of the VLP, e.g., 0.2-50 μg, 0.5-20 μg, 1-10 μg, 1-5 μg, such as about 2, 3, 4, or 5 μg per recipient. Depending on the purpose of the immunization, to stimulate the host's immune response to the target antigen or to desensitize the host's undesirable immune response (i.e., to achieve immunotolerance to an allergen), administration may be repeated once (e.g., for inducing an immune response to the target antigen) or multiple times (e.g., for achieving tolerance to the target allergen) after the initial dose. The repeated dosing of the VLP compositions of this invention is typically carried out in a weekly or biweekly or monthly time interval and may continue for up to 1, 2, 3, 4, or 5 years (for example, monthly administration for a 12-month period) until the allergic symptoms no longer appear upon subsequent exposure to the allergen. In some cases where immunotolerance is desired, the first dose of the VLP compositions is administered after an earlier “pre-treatment” step of exposing the recipient to the suspected allergen at least a week prior (e.g., about 7 to 10 days, or two weeks, or a month prior). For example, an allergen such as the full-length shrimp tropomyosin in the amount of about 0.01-1 mg, about 0.05-0.5 mg, about 0.1-0.2 mg, or about 0.1 mg, optionally along with cholera toxin in the amount of about 1-100 μg, about 5-50 μg, about 2-20 μg, or about 10 μg, is administered to the recipient, e.g., by oral ingestion, as the “pre-treatment” step.

For preparing pharmaceutical compositions of the present invention, various inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., the VLPs of this invention. In tablets, the active ingredient such as the VLPs presenting antigenic epitope(s) of this invention is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound with encapsulating material as a carrier providing a capsule in which the active component (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., the VLPs of this invention) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by suspending the active component (e.g., the VLPs of this invention) in the desired solvent system, and then passing the resulting solution through a membrane filter to sterilize it or, alternatively, by placing the sterilized active component in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between about 3 and 9, more preferably from about 5 to 8, and most preferably from about 6 to 7.

The pharmaceutical compositions of the present invention can be administered for prophylactic and/or therapeutic treatments. In therapeutic applications, compositions are administered to a patient already suffering from a condition in an amount sufficient to prevent, cure, reverse, or at least partially slow or arrest the symptoms of the condition and its complications. An amount adequate to accomplish this is defined as a “therapeutically effective dose.” In prophylactic applications, pharmaceutical compositions of the present invention are administered to a patient susceptible to or otherwise at risk of developing an allergic disease or condition, in an amount sufficient to delay or prevent the onset of the symptoms. Such an amount is defined to be a “prophylactically effective dose.” In either case, amounts effective for therapeutic or prophylactic uses will depend on the (expected) severity of the allergic disease or condition and the weight and general state of the patient, but generally range from about 0.1 μg to about 100 μg of the VLPs per dose for a 70 kg patient, with dosages of from about 0.5 μg to about 10 μg, or from about 1 μg to about 5 μg, e.g., about 2 μg, of the VLPs per dose being more commonly used.

Single or multiple administrations of the compositions can be carried out with dose levels and pattern being selected by the treating physician. In any event, the pharmaceutical formulations should provide a quantity of composition of the present invention sufficient to effectively stimulate or dampen the immune response to the target antigen in the patient, either therapeutically or prophylactically.

### Examples

The following examples are provided by way of illustration only and not by way of limitation. Those of skill in the art will readily recognize a variety of non-critical parameters that could be changed or modified to yield essentially the same or similar results.

This disclosure relates to the method to produce HBcAg-SpyCatcher and its usage to produce polypeptide vaccines for the clinical treatment of shellfish allergy.

### (A) Preparation of Recombinant HBcAg-SpyCatcher

Escherichia coli BL21 (DE3) (Invitrogen) was co-transformed with chaperone plasmid pG-KJE8 (TAKARA) and pET30 (a) vector carrying two copies of HBcAg-149 sequences with SpyCatcher003 (SC) sequences inserted in MIR regions (FIG. 1). Transformed cells were propagated in Auto Induction Medium (Formedium) under induction of 0.5 mg/ml L-Arabinose and I ng/mL tetracycline for expression of His-tagged SC-HBcAg dimer protein at room temperature for 24 hr. Protein purification was then carried out using HisPur Cobalt Spin Column (Thermo Scientific) according to manufacturer's instructions. Protein purity was checked by Sodium dodecyl sulfate-polyacrylamide gel electrophoresis (SDS-PAGE) followed by Simply Blue staining. Ammonium sulphate precipitation was then carried out by mixing 100 μL HBcAg dimer protein, 400 μL assembly buffer (50 mM Tris-HCl, 800 mM NaCl, pH 7.0) and 500 μL 4.32 M saturated ammonium sulphate solution. After agitating for 90 mins under room temperature, the mixture was centrifuged for 30 mins with 15000 rpm to collect the precipitated protein. The protein pellet was then redissolved in assembly buffer and then dialysed by 3500 MWCO dialysis cassette (Thermo Scientific) for 10 days. Dialysed assembled protein was purified by size exclusion chromatography with Enrich SEC 650 column (Bio Rad) on NGC Medium-Pressure Liquid Chromatography Systems (Bio Rad). Accurate detection of assembled SC-HBcAg protein size and shape was done by observing negatively stained (2% uranyl acetate) dialyzed protein on carbon-coated copper grid under transmission electron microscope (TEM) at 80 kV.

SC-HBcAg dimer protein was expressed in E. coli soluble protein. After protein purification, purified SC-HBcAg dimer protein was represented by a bright band at around 67 kDa indicated by the Color Prestained Protein Standard (10-250 kDa) in SDS-PAGE analysis (FIG. 2), which matched with the expected protein size of the SC-HBcAg dimer protein. Each assembled HBcAg particle comprises 90-120 dimers so the size of successfully assembled HBcAg is at least 6030 kDa. In size exclusion chromatography, dialysed SC-HBcAg protein was eluted in void volume that contains protein in size larger than 650 kDa when comparing to the standard with calibration. Under TEM, when comparing to commercial HBcAg control, the SC-HBcAg protein was observed to be integral spherical VLPs with diameter of about 30 nm.

(B) Polypeptides of Tropomyosin with SpyTag Sequences

Eight overlapping polypeptides (P1, P2, P3, P4, P5, P6, P7, and P8) spanning the entire tropomyosin protein (274 amino acids) from Metapenaeus ensis with SpyTag003 sequence fused N-terminal were designed (FIG. 3) and commercially synthesized. Each polypeptide overlaps with two other polypeptides by 10 to 18 amino acids. Each of them contained at least one of the nine B cell epitopes identified by epitope mapping of Met e 1. Each polypeptide contained 56 to 66 amino acids excluding the SpyTag003 sequence.

Based on peptide ELISA, all ST-Polypeptides have lower IgE binding affinity to recombinant tropomyosin (rTM)-specific IgE comparing to rTM protein. Their IgE reactivity was also comparable to that of irrelevant allergens including parvalbumins, enolase and aldolase from salmon.

### (C) Coupling Reaction for HBcAg-Polpeptide Vaccine Production

Ten micromolar SC-HBcAg was reacted with each ST-Polypeptide in excess with specific molar ratio (1:5 for P1 and P2; 1:10 for P6, P7 and P8; 1:30 for P3; 1:50 for P4 and P5) for 3 h at room temperature in a total volume of 50 μL including 5 μL 10× or 16.7 μL 3× reaction buffer (40 mM Na₂HPO₄, 200 mM sodium citrate, pH 6.2). Unconjugated ST-Polypeptides were removed by ultrafiltration using Amicon Ultra-4 with Ultracel-100 regenerated cellulose membrane.

With our design and high throughput plug-and-display system, we have successfully produced eight HBcAg-polypeptides vaccines in our laboratory just by simple mixing. Based on SDS-PAGE, coupling efficiency was maximized by adjusting the reaction ratio to result the least unconjugated or incompletely conjugated SC-HBcAg. Coupling efficiency was shown to be >90% at optimized ratio.

### (D) Immunogenicity of HBcAg-Polypeptide Vaccines

Female BALB/c mice aged 4-5 weeks old were acquired from the Laboratory Animal Services Centre of The Chinese University of Hong Kong. They were subcutaneously immunized on days 0 and 14 with 2 μg of each ST-Polypeptide conjugated SC-HBcAg (eight groups), unconjugated ST-polypeptide (four groups), unconjugated SC-HBcAg, rTM or PBS. Each injection was mixed with 100 μL Adda Vax adjuvant (Invivogen). Blood was collected twice on days 21, 28, 45, 60, and 75 for the determination of IgG and IgG2a antibody levels.

Followed by subcutaneous immunization of BALB/c mice, we neatly demonstrated that (i) HBcAg remarkably promoted the immunogenicity of the polypeptides by 5- to 10-fold (FIG. 5B); (ii) HBcAg specifically promoted IgG2a antibody production, the isotype of human IgG4 in mouse (FIG. 5C); (iii) candidate vaccines HBcAg-P1, HBcAg-P2, HBcAg-P7 and HBcAg-P8 remarkably induced strong tropomyosin-specific IgG and IgG2a responses comparable to tropomyosin (FIG. 5D and FIG. 5E); and (iv) IgG induced by these vaccines inhibited IgE from binding tropomyosin at efficiency of 13.6%-21.8% (FIG. 5F). It is also shown that the antibody response induced by HBcAg-P1 was sustained at day 75 (60 days post-vaccination) and that shows the strong immunogenicity of the vaccine (FIG. 6).

(E) Immunotherapy with HBcAg-Polypeptide Vaccines

3-4 weeks old BALB/c mice were acquired from the Laboratory Animal Services Centre (LASEC) of CUHK. BALB/c mice were divided into eight groups with six mice per group. Mice were sensitized with 0.1 mg rTM and 10 μg cholera toxin in 300 μL PBS each by intragastric gavage on days 0, 12, 19 and 26. One week after the last sensitization, mice were then treated twice at 7-days apart with the HBcAg-P1 at 2 μg and Addavax as adjuvant subcutaneously. Two weeks after treatment, mice were given a high dose 1 mg rTM challenge. Mice in the control group were given equal volume of PBS and Addavax. Immediate allergic responses of the animals were monitored and recorded in a blinded manner after allergen challenge for 40 min. This includes the measurement of rectal temperature and scoring of allergic symptoms. Blood samples were collected to measure serological levels of TM-specific IgE, IgG1 and IgG2a.

As a pilot experiment, BALB/c mice induced with shrimp allergy were treated subcutaneously with 2 μg of HBcAg-P1 twice at bi-weekly interval. It was shown that just two vaccinations are sufficient to protect against allergic reactions (protect against the drop in rectal temperature, FIG. 7A, and diarrhea). Although no significant drop in IgE level was detected, such protection is likely conferred by the remarkably higher IgG2a induced by HBcAg-P1 that block IgE from binding tropomyosin (FIG. 7B). Serum samples were collected from negative control mice (no sensitization/treatment), sham-treated mice (sensitized by shrimp extract and sham-treated with PBS only) and HBcAg-P1-treated mice (sensitized by shrimp extract and treated with HBcAg-P1). The sera were injected intravenously to naïve BALB/c, followed by the intradermal injection of tropomyosin with Evan's blue dye at the back skin. Mice were subsequently sacrificed for the collection of back skin. Our data showed that the presence of IgG2a in HBcAg-P1-treated mice blocked IgE from binding to tropomyosin, as noted by the reduced degranulation and thus leakage of Evan's blue dye at the back skin as compared to that induced by sera of sham-treated mice (FIG. 8).

### (F) Safety of HBcAg-Polypeptide Vaccines

Basophil activation test was performed with fresh blood samples from two shrimp-allergic individuals diagnosed by double-blind, placebo, controlled food challenge. Increasing doses (1-10,000 ng/ml) of HBcAg-P1, un-conjugated HBcAg, shrimp extract and Met e 1 (tropomyosin) were tested using the Flow CAST kit (BÜHLMANN). Note that shrimp extract and Met e 1 induced basophil activation (% CD63+basophil cells) but not HBcAg-P1 and HBcAg even at the highest tested dose (FIG. 9A and FIG. 9B).

MTT assay was performed using PBMCs from healthy donor at 1 million cells/mL stimulated with 1, 12 or 16 μg of HBcAg, HBcAg-P1, Met e 1, positive (0.1% Triton-X) or negative controls (medium only). Data was presented as % viability and note that the vaccines did not trigger cell death (FIG. 9C).

Serum samples were collected from mice (n=4-8 per group) immunized with HBcAg-P1 and mice injected with PBS (negative control) for the measurement of (FIG. 9D) aspartate aminotransferase (AST) and (FIG. 9E) alanine transaminase (ALT) activities. Serum samples from shrimp-sensitized mice treated with HBcAg-P1 were collected on d33 (after first allergen challenge and before treatment), d55 (one week after HBcAg-P1 treatment) and d63 (two weeks after HBcAg-P1 treatment and one day after allergen challenge), and tested for (FIG. 9F) AST and (FIG. 9G) ALT activity. The assays were performed according to the manufacturer's instruction (Abcam; AST: ab105135; ALT: ab105134) with sera diluted at 1:10. Note that both AST and ALT activities remained the same along the experiment, between immunized and non-immunized mice, as well as between sham-treated and HBcAg-P1-treated mice. These indicate no hepatotoxicity induced by the vaccine and thus its safety.

All patents, patent applications, and other publications, including GenBank Accession Numbers, cited in this application are incorporated by reference in the entirety for all purposes.

SEQUENCE LISTING

SEQ ID NO: 1 (amino acid sequence of a truncated HBcAg protein 149: bold/italicized =
linker sequence; underline = SpyCatcher sequence)
MDIDPYKEFGATVELLSFLPSDFFPSVRDLLDTASALYRDALESPEHASPHHTALRQAILC
WGELMTLATWVGVNLED78GGGSDSATHIKFSKRDEDGRELAGATMELRDSSGKTISTW
ISDGHVKDFYLYPGKYTFVETAAPDGYEVATPIEFTVNEDGQVTVDGEATEGDAHTGG
GSP⁷⁹ASRDLVVSYVNTNMGLKFRQLLWFHISALTFGRETVIEYLVSFGVWIRTPPAYRPP
NAPILSTLPETTVV¹⁴⁹GGSGGSGGSGGSGGSMDIDPYKEFGATVELLSFLPSDFFPSVRDL
LDTASALYRDALESPEHASPHHTALRQAILCWGELMTLATWVGVNLED⁷⁸GGGSDSATH
IKFSKRDEDGRELAGATMELRDSSGKTISTWISDGHVKDFYLYPGKYTFVETAAPDGYE
VATPIEFTVNEDGQVTVDGEATEGDAHTGGGSP⁷⁹ASRDLVVSYVNTNMGLKFRQLLWF
HISALTFGRETVIEYLVSFGVWIRTPPAYRPPNAPILSTLPETTVV
SEQ ID NO: 2 (amino acid sequence of shrimp tropomyosin protein)
MKLEKDNAMDRADTLEQQNKEANNRAEKSEEEVHNLQKRMQQLENDLDQVQESLLK
ANNQLVEKDKALSNAEGEVAALNRRIQLLEEDLERSEERLNTATTKLAEASQAADESER
MRKVLENRSLSDEERMDALENQLKEARFLAEEADRKYDEVARKLAMVEADLERAEER
AETGESKIVELEEELRVVGNNLKSLEVSEEKANQREEAYKEQIKTLTNKLKAAEARAEF
AERSVQKLQKEVDRLEDELVNEKEKYKSITDELDQTFSELSGY
SEQ ID NO: 3 (amino acid sequence of P1 epitope: double underlined/italicized = linker
sequence; bold = IgE-binding epitope; underlined = T cell epitope)
RGVPHIVMVDAYKRYKGGGSMKLEKDNAMDRADTLEQQNKEANNRAEKSEEEVHNL
QKRMQQLENDLDQV
SEQ ID NO: 4 (amino acid sequence of P2 epitope: double underlined/italicized = linker
sequence; bold = IgE-binding epitope; underlined = T cell epitope)
RGVPHIVMVDAYKRYKGGGSVHNLQKRMQQLENDLDQVQESLLKANNQLVEKDKA
LSNAEGEVA
SEQ ID NO: 5 (amino acid sequence of P3 epitope: double underlined/italicized = linker
sequence; bold = IgE-binding epitope; underlined = T cell epitope)
RGVPHIVMVDAYKRYKKGGGSALSNAEGEVAALNRRIQLLEEDLERSEERLNTATTKL
AEA
SEQ ID NO: 6 (amino acid sequence of P4 epitope: double underlined/italicized = linker
sequence; bold = IgE-binding epitope; underlined = T cell epitope)
RGVPHIVMVDAYKRYKKGGGSNTATTKLAEASQAADESERMRKVLENRSLSDEERMDA
LENQLKEARFL
SEQ ID NO: 7 (amino acid sequence of P5 epitope: double underlined/italicized = linker
sequence; bold = IgE-binding epitope; underlined = T cell epitope)
RGVPHIVMVDAYKRYKKGGGSNQLKEARFLAEEADRKYDEVARKLAMVEADLERAEE
RAETGESKI
SEQ ID NO: 8 (amino acid sequence of P6 epitope: double underlined/italicized = linker
sequence; bold = IgE-binding epitope; underlined = T cell epitope)
RGVPHIVMVDAYKRYKKGGGSERAETGESKIVELEEELRVVGNNLKSLEVSEEKANQR
EEA
SEQ ID NO: 9 (amino acid sequence of P7 epitope: double underlined/italicized = linker
sequence; bold = IgE-binding epitope; underlined = T cell epitope)
RGVPHIVMVDAYKRYKKGGGSEKANQREEAYKEQIKTLTNKLKAAEARAEFAERSVQK
LQKEVDR
SEQ ID NO: 10 (amino acid sequence of P8 epitope: double underlined/italicized = linker
sequence; bold = IgE-binding epitope; underlined = T cell epitope)
RGVPHIVMVDAYKRYKKGGGSAERSVQKLQKEVDRLEDELVNEKEKYKSITDELDQTF
SELSGY
SEQ ID NO: 11 (amino acid sequence of exemplary SpyCatcher peptide)
SATHIKFSKRDEDGRELAGATMELRDSSGKTISTWISDGHVKDFYLYPGKYTFVETAAP
DGYEVATPIEFTVNEDGQVTVDGEATEGDAHT
SEQ ID NO: 12 (amino acid sequence of exemplary SpyTag peptide)
RGVPHIVMVDAYKRYK

1. A method for making an antigen epitope-presenting virus-like particle (VLP), comprising:
(1) forming a VLP with a modified viral protein comprising at least a portion of the viral protein fused to a first partner peptide; and
(2) reacting the VLP with a fusion antigenic peptide comprising an epitope of a target antigen fused with a second partner peptide to form an isopeptide bond between the first and second partner peptides and yield a VLP presenting the epitope of the target antigen.

2. The method of claim 1, wherein the viral protein is a Hepatitis B virus core protein (HBcAg).

3. The method of claim 1, wherein the target antigen is a shrimp tropomyosin.

4. The method of claim 3, wherein the epitope is any one of P1 to P8.

5. The method of claim 1, wherein step (2) comprises reacting the VLP with two or more fusion antigenic peptides, each comprising a different epitope fused with a second partner peptide.

6. The method of claim 5, wherein in step (2) the two or more fusion antigenic peptides each comprise a different epitope independently selected from P1 to P8.

7. The method of claim 1, wherein the first and second partner peptides comprise a SpyCatcher peptide and a SpyTag peptide.

8. An antigen epitope-presenting VLP made by the method of claim 1.

9. A composition comprising the VLP of claim 8 and a pharmaceutically or physiologically acceptable excipient.

10. The composition of claim 9, further comprising an adjuvant.

11. The composition of claim 9 or 10, which is formulated for injection.

12. A method for eliciting an immune response in an individual, comprising administering to the individual an effective amount of the composition of claim 9.

13. The method of claim 12, wherein the administering comprises injection of the composition.

14. The method of claim 13, wherein the injection is subcutaneous injection.

15. The method of claim 12, wherein the immune response is a B cell and/or T cell response against the target antigen.

16. The method of claim 12, wherein the immune response is tolerance for the target antigen.

17. The method of claim 12, wherein the administering is repeated at least once.

18. The method of claim 16, further comprising, prior to the administering, contacting the recipient with the target antigen to sensitize the recipient to the antigen.

19. The method of claim 18, wherein the administering is repeated at least twice at a weekly or bi-weekly interval following the contacting.

20. The method of claim 19, wherein the individual is previously diagnosed or is suspected to suffer from an allergy to the target antigen.

21. The method of claim 20, wherein the individual is previously diagnosed or is suspected to suffer from a shellfish allergy, and wherein the individual is first contacted with a shrimp tropomyosin prior to being administered the composition.

22. A kit comprising the composition of claim 9.

23. The kit of claim 22, wherein the composition is a vaccine against a shellfish allergy.

24. The kit of claim 23, wherein the vaccine is adapted for injection.

25. The kit of claim 22, further comprising an instruction manual.
